(19) United States
(12) Patent Application Publication
(10) Pub. No.: US 2012/0134947 A1
(43) Pub. Date: May 31, 2012

(54) ANTIPERSPIRANT COMPOSITIONS AND PRODUCTS HAVING COOLING SENSATION EFFECTS AND METHODS FOR MAKING THE SAME

(75) Inventors: Travis T. Yarlagadda, Phoenix, AZ (US); Thomas Doering, Scottsdale, AZ (US)

(73) Assignee: The Dial Corporation, Scottsdale, AZ (US)

(21) Appl. No.: 12/956,189

(22) Filed: Nov. 30, 2010

(51) Int. Cl. A61K 8/97; B29C 39/12; A61Q 15/00

(52) U.S. Cl. 424/65; 264/255

(57) ABSTRACT

A personal care product comprises an antiperspirant product that is housed within a container. The antiperspirant product includes a first portion comprising a dispersed cooling sensation agent. A second portion has a composition different from the first portion and comprises a solubilizer.

### FIELD OF THE INVENTION

The present invention generally relates to personal care antiperspirant compositions, products, and methods for making the same, and more particularly relates to antiperspirant compositions that exhibit antiperspirant efficacy and cooling sensation effects, antiperspirant products comprising such antiperspirant compositions, and methods for manufacturing such antiperspirant compositions and products.

### BACKGROUND OF THE INVENTION

Antiperspirant and deodorant compositions are well known personal care products used to prevent or eliminate sweat and body odor caused by sweat. The compositions come in a variety of forms and may be formulated, for example, into aerosols, pumps, sprays, liquids, roll-ons, lotions, creams, and sticks (both hard and soft), etc.

There are various types of stick antiperspirant compositions that are desirable by large majority of the population because of their ease of application and the presence of active antiperspirant compounds, e.g. antiperspirant salts, which prevent or block the secretion of sweat and its accompanying odors. In one type, an antiperspirant salt is suspended in an anhydrous vehicle often including a solid water-insoluble wax. In a second type, an antiperspirant salt is dissolved in a liquid vehicle such as propylene glycol and gelled with a gelling agent such as dibenzylidene sorbitol. A third type includes an emulsion of an aqueous phase containing the antiperspirant salt and an oil phase containing, for example, a volatile silicone, fragrances, gellants, and other additives.

Stick antiperspirant products include an antiperspirant composition within a container. During use of the product, the top of the container is removed and the application surface of the composition is contacted with the underarm by swiping or rubbing the stick across the skin. Sometimes the product also includes an undercap, or factory seal, covering the application surface that is removed prior to first use. During use, some of the composition is transferred to the skin, and a container generally also includes some mechanism for moving the composition upwards through the container to continue to provide an exposed application surface.

Commercial markets for antiperspirant and deodorant products are highly competitive with consumers wanting increased antiperspirant efficacy and refreshing effects from these products. One problem for antiperspirant and deodorant manufacturers is that the ingredients and/or additives used in the product for antiperspirant efficacy and refreshing effects may have limited shelf life and become less effective and/or less refreshing over time. Another problem is that such antiperspirant or deodorant compositions may not provide efficacy and refreshing effects when the user needs it most throughout the day.

Accordingly, it is desirable to provide antiperspirant products that exhibit strong antiperspirant efficacy and refreshing effects which have good shelf life and/or are more responsive to the user when needed most. In addition, it is desirable to provide methods for manufacturing such antiperspirant products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products and methods for manufacturing antiperspirant products are provided herein. In accordance with an exemplary embodiment, a personal care product is provided. The personal care product comprises a container. An antiperspirant product is housed within the container and comprises a first portion comprising a dispersed cooling sensation agent. A second portion that is in non-mutual spatial relationship with the first portion and has a composition different from the first portion and includes a solubilizer. The solubilizer is effective to solubilize the dispersed cooling sensation agent within body generated moisture and thereby impart to a user a cooling sensation effect.

In accordance with another exemplary embodiment, an antiperspirant product comprises a first portion comprising a dispersed cooling sensation agent adsorbed on surfaces and absorbed into pores of a first plurality of silica particles that are dispersed throughout the first portion. A second portion that is in non-mutual spatial relationship with the first portion and has a composition different from the first portion. The second portion comprises a solubilizer adsorbed on surfaces and absorbed into pores of a second plurality of silica particles that are dispersed throughout the second portion. The solubilizer is effective to solubilize the dispersed cooling sensation agent within body generated moisture and thereby impart to a user a cooling sensation effect. The first and second pluralities of silica particles are effective to release the dispersed cooling sensation agent and the solubilizer when contacted with body generated moisture.

In accordance with a further exemplary embodiment, a method for manufacturing an antiperspirant product is provided. The method comprises depositing a first portion of the antiperspirant product into a mold and allowing the first portion to at least partially solidify. The first portion comprises a dispersed cooling sensation agent. A second portion of antiperspirant product is deposited into the mold and the second portion is allowed to at least partially solidify. The second portion has a composition different from the first portion and comprises a solubilizer effective to solubilize the dispersed cooling sensation agent within body generated moisture and thereby impart to a user a cooling sensation effect.

### BRIEF DESCRIPTION OF THE DRAWINGS

The present invention will hereinafter be described in conjunction with the following drawing figures, wherein like numerals denote like elements, and

FIG. 1 is a perspective view of a personal care antiperspirant product in accordance with an exemplary embodiment;

FIG. 2 is a plan view of the antiperspirant product of FIG. 1;

FIG. 3 is an exploded perspective view of the antiperspirant product of FIG. 1 in accordance with an exemplary embodiment;

FIG. 4 is a perspective view showing the initial step of a filling assembly being inserted into a container in a process for manufacturing the antiperspirant product of FIG. 3 in accordance with an exemplary embodiment;

FIG. 5 is a perspective view of an outer nozzle assembly of the filling apparatus shown in FIG. 4;

FIGS. 6 and 7 are cross-sectional views of the center nozzle tube shown in FIG. 5;

FIG. 8 is an enlarged detailed view of a portion of the outer nozzle assembly of FIG. 5;

FIG. 9 is a perspective view of an inner nozzle assembly of the filling apparatus shown in FIG. 4;

FIG. 10 is a perspective view of the inner nozzle assembly shown in FIG. 9, with one half of the nozzle housing removed to show the interior of the inner nozzle assembly;

FIG. 11 is a side view of the inner nozzle assembly shown in FIG. 10 and FIG. 12 is an exploded view showing internal components of the inner nozzle assembly;

FIG. 13 is a perspective view showing a further step in the manufacturing process of FIG. 4;

FIG. 14 is a cross-sectional view taken along line 14-14 in FIG. 13;

FIG. 15 is a perspective view showing a further step in the manufacturing process of FIGS. 4 and 13;

FIG. 16 is a cross-sectional view taken along line 16-16 in FIG. 15;

FIG. 17 is a perspective view showing a further step in the manufacturing process of FIGS. 4, 13, and 15;

FIG. 18 is a cross-sectional view taken along line 18-18 in FIG. 17;

FIG. 19 is a partially exploded perspective view of a portion of a nozzle assembly used in an alternative process for manufacturing the antiperspirant composition illustrated in FIG. 3 in accordance with an exemplary embodiment;

FIG. 20 is a fully exploded perspective view of the nozzle assembly of FIG. 19;

FIG. 21 is a cross sectional view showing the nozzle assembly of FIG. 19 filling a container; and

FIG. 22 is a flowchart of an example of a process for manufacturing an antiperspirant composition and product in accordance with the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to personal care antiperspirant products that exhibit antiperspirant efficacy and provide a cooling sensation effect to a user upon perspiration. The antiperspirant product comprises two distinct portions, each having a different composition. One or both portions may have an active antiperspirant ingredient or ingredients but a first portion has a cooling sensation agent that is dispersed throughout that portion, while a solubilizer is dispersed throughout the remaining second portion. According to one exemplary embodiment, the cooling sensation agent is captured/carried on a plurality of silica particles that are dispersed throughout the first portion. Likewise, the solubilizer is captured on another plurality of silica particles that are dispersed throughout the second portion. When the antiperspirant product is applied to an underarm, for example, the two portions of the antiperspirant product are combined. Preferably, the silica particles are porous and hydrophilic such that when the user perspires, the perspiration is readily absorbed by the silica particles, displacing or driving out the cooling sensation agent and the solubilizer from the silica particles. As such, the cooling sensation agent and solubilizer are free to interact such that the solubilizer solubilizes the cooling sensation agent. The solubilized cooling sensation agent intermingles with the perspiration, producing a cooling sensation effect that is perceived by the user. The inventors have unexpectedly found that by keeping the cooling sensation agent and the solubilizer separated from each other prior to application and further, by allowing the components to combine upon exposure to perspiration, the antiperspirant product has an excellent shelf-life and provides the user a cooling sensation effect throughout the day when it is most needed, e.g., when the user perspires.

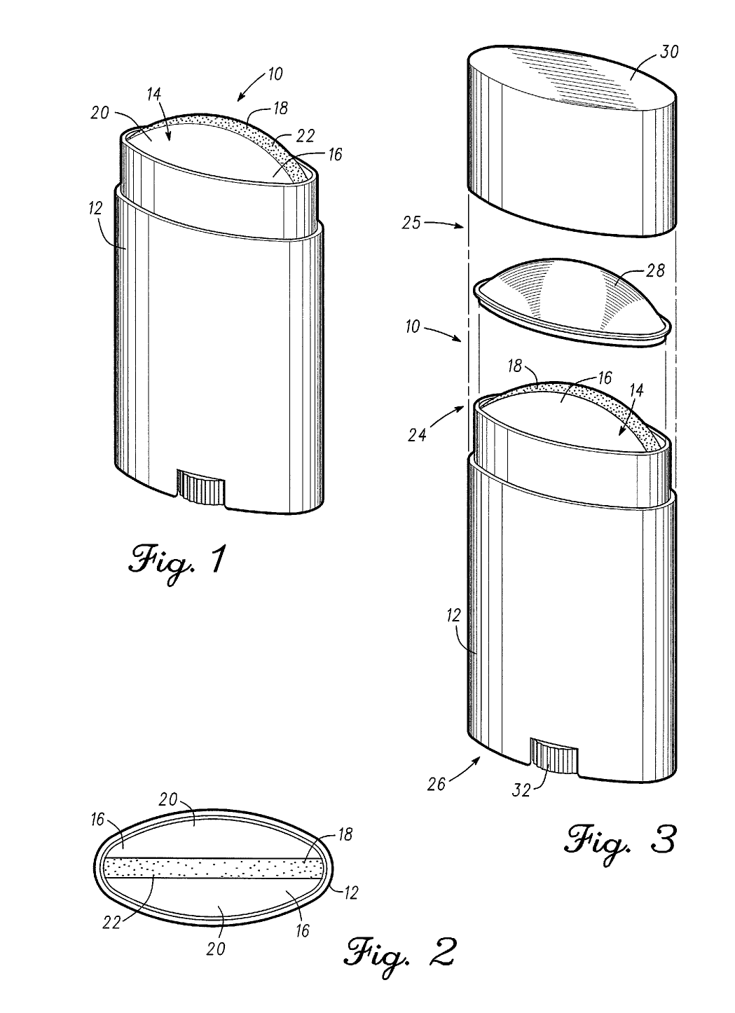

Referring to FIGS. 1 and 2, a personal care antiperspirant product 10 in accordance with an exemplary embodiment comprises a first portion 16 and a second portion 18. The term “portion,” as used herein, includes the section or sections of the antiperspirant product having the same composition; for example, two sections having the same composition but separated by a third section (for example, a central stripe) having a different composition constitute a single “portion.” The first portion 16 may have a color different from that of second portion 18 or the portions may be of the same color. As shown, the first and second portions 16 and 18 are visibly separated and are in non-mutual spatial relationship with each other. The antiperspirant product 10 has an application surface 14 that is substantially dome-shaped and that is configured to be applied to skin, such as for example, an underarm. The antiperspirant product 10 also may comprise a container or dispenser 12 for dispensing first portion and second portion 16 and 18 to the skin.

In one exemplary embodiment, the first portion 16 is an outer portion and the second portion 18 is an inner portion and the application surface 14 comprises a surface 20 of first portion 16 that is bisected by an adjacent surface 22 of the second portion 18. The first portion 16 and the second portion 18 are preferably solid wax formulations, however, other suitable antiperspirant formulations may be used for either or both of the portions 16 and 18. The first portion 16 is positioned on outside regions of the antiperspirant product 10 while the second portion 18 is positioned between the regions of the first portion 16. This configuration is a preferred configuration because the first portion 16 has a melting point that is higher than the melting point of the second portion 18. During manufacture, as described in more detail below, the second portion 18 is formed after the first portion 16 and thus, will not melt the already prepared first portion 16. However, it will be appreciated that the invention is not limited to the configuration of the first portion 16 and the second portion 18 illustrated in FIGS. 1 and 2. Rather, it will be appreciated that the second portion 18 can be the outer portion and the first portion 16 can be the inner portion.

It will be appreciated that the first portion 16 and the second portion 18 may also have other configurations. For example, the second portion 18 may be completely surrounded by the first portion 16 or vice versa. Alternatively, rather than forming one strip bisecting the first portion 16, the second portion 18 may form two or more strips. The antiperspirant product 10 may also comprise a third portion and other additional portions that do not comprise the compositions of the first portion 16 and the second portion 18. The first portion 16 and the second portion 18 may take any other configuration suitable for applying the portions to skin. The surface 20 of first portion 16 and the surface 22 of the second portion 18 each comprises at least 15%, and preferably at least 25%, of the application surface 14. Each surface 20 and 22 may even comprise, for example, at least 40% of the application surface 14.

In one exemplary embodiment, the first and second portions 16 and 18 are made of formulations having different compositions. The first portion 16 contains a cooling sensation agent and the second portion 18 has a solubilizer that is effective to solubilize the cooling sensation agent when contacted with the dispersed cooling sensation agent. Preferably, the cooling sensation agent is dispersed throughout the first portion 16 and not the second portion 18, and the solubilizer is dispersed throughout the second portion 18 and not the first portion 16. By keeping the cooling sensation agent and the solubilizer separated into their respective portions 16 and 18, the inventors have found that the efficacy of the cooling sensation agent is better maintained.

Non-limiting examples of cooling sensation agents are menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, or a mixture thereof. In one example, the cooling sensation agent is Winsense® Extra 500, which comprises a mixture of ethyl menthane carboxamide, menthyl diisopropyl propionamide and menthyl lactate. Winsense® Extra 500 is manufactured by LyondellBasell of Rotterdam, the Netherlands. Other suitable cooling sensation agents known to those skilled in the art may also be used. The first portion 16 preferably contains an amount of the cooling sensation agent in the range of from about 0.50 to about 3.0 weight percent (wt. %). The term “about” as used herein means within typical manufacturing tolerances. Non-limiting examples of solubilizers are polyglycol, polyethylene glycol, polypropylene glycol, or a mixture thereof. Other suitable solubilizers known to those skilled in the art may also be used. The second portion 18 preferably contains an amount of the solubilizer in the range of from about 0.7 to about 8.5 wt. %.

In one exemplary embodiment, the cooling sensation agent is captured or adhered to the surfaces of a first plurality of silica particles that are in the form of a solid powder that is dispersed throughout the first portion 16. As such, the cooling sensation agent is effectively dispersed throughout the first portion 16. Preferably, the silica particles are porous and hydrophilic. In one example, the silica particles are precipitated silica particles. One such type is Sipernat® 22S, which is manufactured and sold by Evonik Industries of Mobile, Ala. and is comprised of fine silica particles with high absorption capacity for liquids, especially water. The cooling sensation agent is adsorbed onto, absorbed into and effectively captured by the porous hydrophilic silica particles. When the porous hydrophilic silica particles are exposed to an amount of moisture, e.g. perspiration, the moisture is readily taken up by the porous hydrophilic silica particles displacing and releasing (e.g. eluting) the cooling sensation agent from the particles. Preferably, the first portion 16 comprises the first plurality of silica particles in an amount of from about 1.0 to about 4.0 wt. %. Other suitable silica particles or hydrophilic carriers known to those skilled in the art may also be used to disperse the cooling sensation agent throughout the first portion 16.

In another exemplary embodiment, the solubilizer is captured or adhered to the surfaces of a second plurality of silica particles that are in the form of a solid powder and that are preferably porous and hydrophilic. Thus, the solubilizer is also effectively dispersed throughout the second portion 18 as particles of the solid powder. The second plurality of silica particles may be the same type as the first plurality of silica particles or, alternatively, the first and second plurality of silica particles may be different types of silica. In one example, the solubilizer is adsorbed onto, absorbed into and effectively captured by the porous hydrophilic silica particles. When the porous hydrophilic silica particles are exposed to an amount of moisture, e.g. perspiration, the moisture is readily taken up by the porous hydrophilic silica particles displacing and releasing (e.g. eluting) the solubilizer from the particles. Preferably, the second portion 18 comprises the second plurality of silica particles in an amount of from about 0.5 to about 3.0 wt. %. In an exemplary embodiment, the first and second pluralities of silica particles are hydrophilic forms of precipitated silica having a BET surface area of from about 50 to about 1000 m²/g, preferably from about 100 to about 700 m²/g, and most preferably from about 150 to about 600 m²/g; a particle size d₅₀, determined by laser diffraction, from about 2 to about 130 μm, preferably from about 3 to about 20 μm; a DOA (dioctyl acetate) absorption from about 150 to about 400 g DBP/100 g silica, preferably from about 200 to about 350 g DBP/100 g silica.

Additionally, one or both of the first and second portions 16 and 18 may contain a relatively low amount of an unbound cooling sensation agent and an unbound solubilizer that are not captured on either one of the first and second plurality of silica particles. The unbound cooling sensation agent is intermingled with and solubilized by the unbound solubilizer to provide the cooling sensation effect when the antiperspirant product is initially applied to the user and for some period throughout the day thereafter.

Either one or both of the formulations that form the first and second portions 16 and 18 may comprise an anhydrous, hydrophobic vehicle, which includes a volatile silicone and/or high melting component, and an active antiperspirant compound suspended in the anhydrous, hydrophobic vehicle. In one exemplary embodiment, the same active antiperspirant compound or compounds is used in both the first portion and the second portion. Alternatively, the second portion 18 may comprise a different active antiperspirant compound or compounds than the first portion 16. The active antiperspirant compound is preferably in a perspiration-reducing effective amount. At least one and preferably both portions 16 and 18 comprise an active antiperspirant compound.

The active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration can not diffuse to the skin surface. Some active antiperspirant compounds that may be used in the first portion include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium pentachlorohydrex. In another embodiment, the first portion 16 and/or the second portion 18 comprise an active antiperspirant compound present in the amount of 0 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycerin. In a more preferred embodiment, the antiperspirant compound comprises aluminum zirconium pentachlorohydrex at an active level of about 18.0 to about 24.0 wt. % (USP).

The high melting components may include any material suitable for use in an antiperspirant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyall kylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant product 10 contemplated herein also may comprise additives, such as those used in conventional antiperspirants. For example, in addition to, or instead of, antiperspirant efficacy, the first and/or second portion 16 and 18 may comprise additives that cause the antiperspirant product to exhibit long-lasting fragrance, odor protection, bacteria control, and/or another desired purpose and/or function. These additives include, but are not limited to, fragrances, including encapsulated fragrances, dyes, pigments, preservatives, antioxidants, moisturizers, and the like. These optional ingredients can be included in the first and/or second portion 16 and 18 in an amount of 0 to about 20 wt. %.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silica, fumed silica, particulate polyolefins, talcum materials, colorants and preservatives may also be included as desired. For example, the composition may include up to about 10% fragrance or about 2% colorant by weight.

As noted above, in addition to, or instead of, an active antiperspirant compound, the first portion and/or the second portion 16 and 18 may comprise a component or components that cause the first portion and/or the second portion 16 and 18 to exhibit or impart a desired function or purpose in addition to, or instead of, antiperspirant efficacy. For example, the second portion 18 may comprise deodorant active ingredients. A suitable deodorant active ingredient is any agent that inhibits, suppresses, masks or neutralizes malodor. These may include (1) antimicrobial or bactericidal agents that kill the bacteria responsible for malodor production, (2) agents that inhibit or suppress or interfere with the bacterial enzymatic pathway that produces malodor, and (3) agents that mask or absorb or neutralize malodor. “Fragrances” as used herein are not considered deodorant active ingredients. Examples of deodorant actives ingredients include triclosan, triclocarban, usnic acid salts, zinc phenolsulfonate, b-chloro-D-alanine, D-cycloserine, animooxyacetic acid, cyclodextrine, and sodium bicarbonate. Alternatively, or in addition, the portions may comprise fragrances, for example, in an amount that imparts a long-lasting fragrance to the antiperspirant product.

In accordance with exemplary embodiments, a method for manufacturing the antiperspirant product illustrated in FIGS. 1 and 2 is shown in FIGS. 3-19 and 22. With reference to FIG. 22, the method generally denoted at 210 comprises mixing a cooling sensation agent and silica particles to form a cooling sensation agent premix (step 216). In one example, the cooling sensation agent is pre-blended with water and special denatured alcohol (SD) prior to being mixed with the silica particles. The cooling sensation agent blend preferably comprises by weight from about 50 to about 80% cooling sensation agent, from about 5 to about 15% water and from about 10 to about 25% alcohol (SD). The cooling sensation agent blend is then mixed with the first plurality of silica particles (e.g. porous hydrophilic silica particles) preferably using a low shear mixing process until the cooling sensation agent blend has been absorbed homogeneously (e.g. adsorbed onto and into the pores) into the silica particles. A hold time may be used after mixing to allow for a more homogeneous blend of the premix while preventing over-mixing, which could lead to some breakdown of the silica particles. In one embodiment, the cooling sensation agent premix comprises by weight from about 50 to about 80%, preferably from about 60 to about 75%, of the cooling sensation agent blend and from about 25 to about 40% of the silica particles. Without being limited by theory, it is believed that the water and alcohol used in the cooling sensation agent blend facilitates adsorption of the cooling sensation agent onto the porous walls of hydrophilic silica particles during mixing. Mixers that work well are plow shear mixers, conical blade mixers or by hand for lab scale batches. Other suitable mixers known to those skilled in the art may be used.

An active antiperspirant compound is mixed with a structurant to form a first antiperspirant premix (step 220). Other ingredients including hot melt waxes, etc. also can be added during or after formation of the first antiperspirant premix. The cooling sensation agent premix is then combined with the antiperspirant premix (step 222). The mixing disperses the silica particles, which have the cooling sensation agent captured thereon, throughout the first portion.

A solubilizer and a second plurality of silica particles are combined to form a solubilizer agent premix (step 228). In one example, the solubilizer is pre-blended with water prior to being mixed with the silica particles. The solubilizer blend comprises by weight from about 50 to about 80% solubilizer and from about 20 to about 50% water. The solubilizer blend is then mixed with the silica particles (e.g. porous hydrophilic silica particles) preferably using a low shear mixing process until the liquids have been absorbed homogeneously (e.g. adsorbed onto and into the pores) into the silica particles. A hold time may additionally be used after mixing to allow for a more homogeneous blend of the premix while preventing over-mixing which could lead to some breakdown of the silica particles. In one embodiment, the solubilizer premix comprises by weight from about 50 to about 80%, preferably from about 60 to about 75%, of the solubilizer blend and from about 25 to about 40% of the silica particles. Again without being limited by theory, it is believed that the water used in the solubilizer blend facilitates adsorption of the cooling sensation agent onto the porous walls of hydrophilic silica particles during mixing. Similar mixers as those mentioned above or any other suitable mixer may be used to form a solubilizer agent premix.

A second antiperspirant premix is formed (step 232). The second antiperspirant premix may comprise an active antiperspirant compound that is the same as or different from any active antiperspirant compound present in the first antiperspirant premix or may comprise no active antiperspirant compound at all. The second antiperspirant premix comprises a structurant and any additional ingredients desired for a particular composition application. The solubilizer agent premix and the second antiperspirant premix then are combined to form the second portion (step 234). The second portion is mixed so that the silica particles upon which the solubilizer is captured are dispersed therethrough. While the above description discusses the formation of the first portion before the formation of the second portion, it will be understood that the invention is not so limited and that the second portion may be formed before or during formation of the first portion.

As discussed in further detail below with reference to FIGS. 3-21, the antiperspirant product is molded directly into a mold by depositing the first portion in molten form into the mold (step 236) and at least partially solidifying the first portion (step 238), and depositing the second portion in molten form into the mold (step 239) and at least partially solidifying the second portion (step 240) to form the antiperspirant product. The mold may be used as the container for the antiperspirant product to form the antiperspirant composition 25 illustrated in FIG. 3. It will be appreciated, however, that the invention is not limited to use of the container as a mold and that any satisfactory mold may be used for manufacturing the antiperspirant product.

Referring to FIGS. 3 and 22, the container 12 has an application end 24 and an opposite end 26. The container 12 also contains a factory seal 28, which is placed over the application surface 14 of antiperspirant product 10 to protect it during shipment and to render it tamper-proof prior to purchase, and a cover 30. The factory seal 28 is removed by the user, and the cover is used during storage of the product between uses. As the product is exhausted, it is advanced from the container by the user using advancement device 32, e.g., a screw mechanism as shown, at opposite end 26 of container 12.

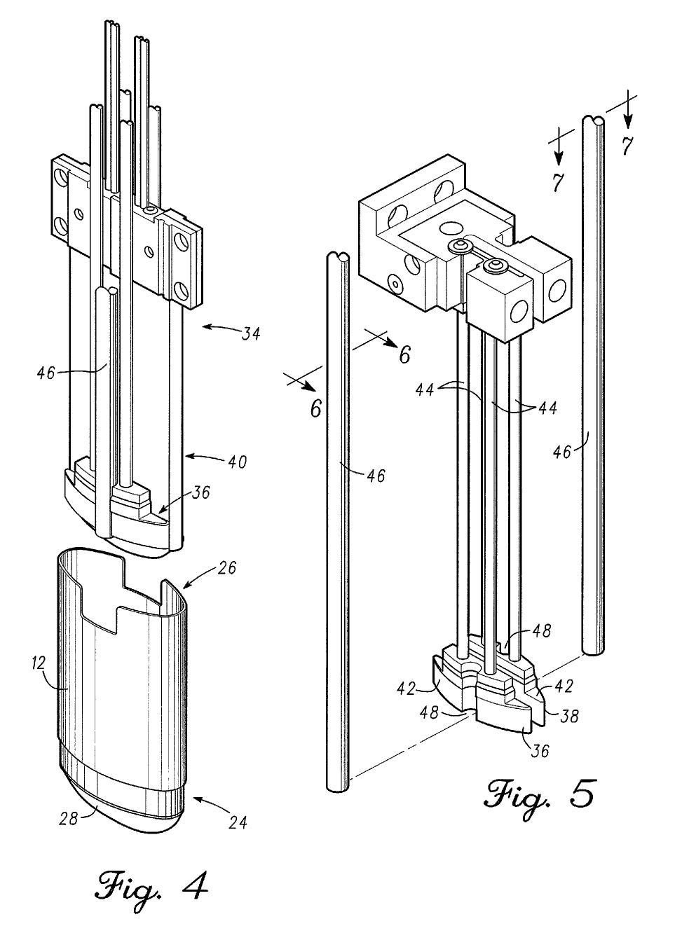

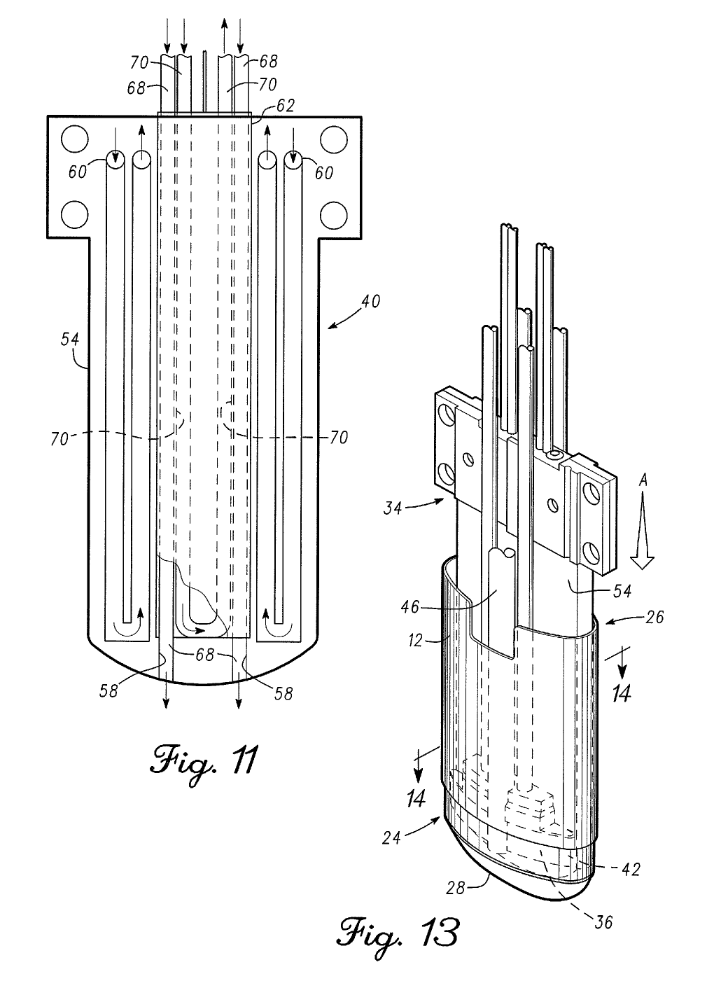

Referring to FIG. 4, a filling assembly 34 is positioned above opposite end 26 of an empty container 12. The factory seal 28 is in place, sealing the application end 24 of the container 12. The filling assembly 34 is lowered into the container 12 and is used to fill two compositions into the container, as will be described below with reference to FIGS. 13-18. The filling assembly 34 will first be described herein.

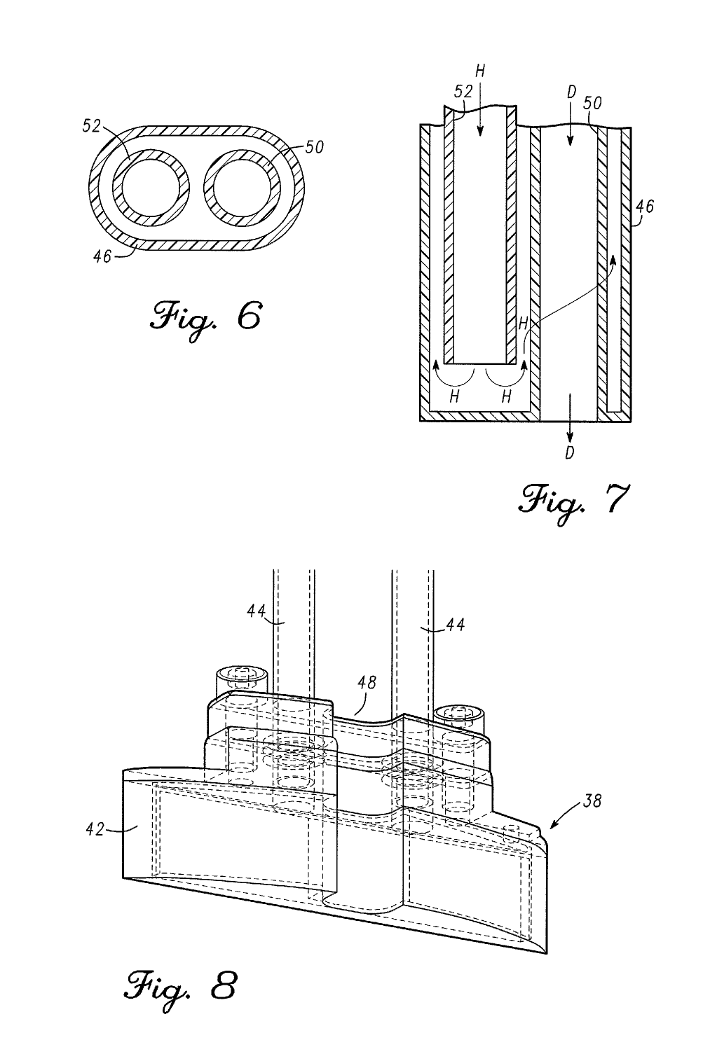

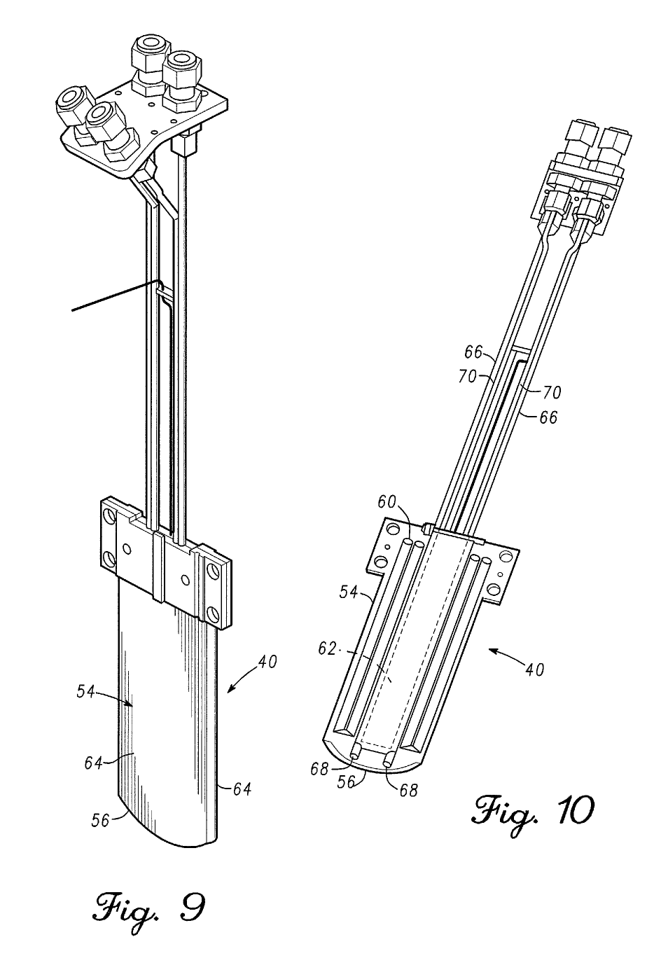

The components of filling assembly 34 are shown individually and in detail in FIGS. 5-10. The filling assembly consists of two outer nozzle assemblies 36 and 38, as shown in FIG. 5, and an inner nozzle assembly 40, as shown in FIG. 9. A first portion of the antiperspirant product is delivered by the outer nozzle assemblies 36 and 38, and a second portion is delivered by the inner nozzle assembly 40. The term “nozzle” as used herein refers to any device that is capable of delivering a fluid composition.

Each of the outer nozzle assemblies 36 and 38, one of which is shown in detail in FIG. 8, include a scraper body 42 that is mounted on two outer tubes 44. The scraper body is hollow, and is chilled by the circulation of cooling media. Its function will be discussed below. A center nozzle tube 46, disposed between the outer tubes 44, is retained in a groove 48 (FIG. 8) in the scraper body (center nozzle tube 46 is omitted in FIG. 8 for clarity). The two outer tubes 44 support the scraper body 42, allowing it to be moved vertically, and circulate cooling media to and from the scraper body. As shown in FIGS. 6 and 7, the center nozzle tube 46 consists of a delivery tube 50 and a heating tube 52. The heating tube circulates heating media (arrows H, FIG. 7) to maintain the first portion in a molten state as it is delivered thought the delivery tube 50 (arrows D, FIG. 7). A temperature sensor, e.g., a thermocouple, thermistor, or the like (not shown), may be provided on one or both of the scraper bodies to measure the temperature of the scraper body.

The inner nozzle assembly 40 includes a housing 54 that provides a molding surface for the first portion, as will be discussed below. The housing 54 includes a curved leading edge 56 shaped to sealingly engage the inner surface of the factory seal 28. If desired, the leading edge 56 may be a relatively sharp edge to provide a concentrated pressure against the factory seal 28. The interior of the housing 54 is shown in FIGS. 10, 11, and 12. The housing 54 defines a pair of delivery channels 58, a pair of substantially U-shaped cooling channels 60, and a central rectangular channel 62. The cooling channels circulate cooling media to chill the outer surface 64 of housing 54. The central rectangular channel 62 receives an assembly 66, shown in FIG. 12, which includes a pair of delivery tubes 68 brazed to a substantially U-shaped heating tube 70. The heating tube 70 circulates heating media to heat the second phase as it is being delivered through the delivery tubes 68. The assembly 66 is wrapped in insulation 72 (FIG. 12) to insulate it within the chilled housing. The inner nozzle assembly 40 may also include one or more temperature sensors (not shown) to determine the heating and/or cooling temperatures.

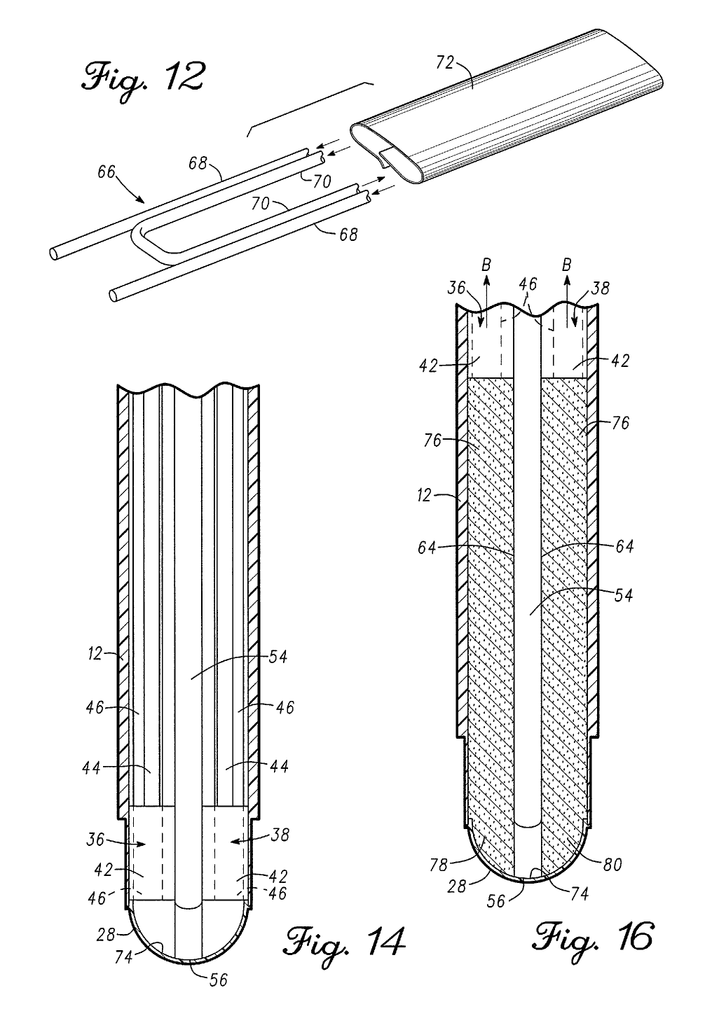

The process of filling the container 12, and thus molding the antiperspirant product 10, is shown in FIGS. 13-18. First, as shown in FIGS. 13 and 14, the filling assembly 34 is inserted into the container 12, through the opposite end 26 (arrow A), until the leading edge 56 of the housing 54 contacts the inner surface 74 of the factory seal 28. At this stage of the process, both the inner nozzle assembly 40 and the outer nozzle assemblies 36 and 38 are fully extended into the container 12. Although the cover 30 is omitted in FIGS. 4 and 13-18, for clarity, the cover is in place during the molding process. Cover 30 provides a flat surface on which the container can rest during filling, and also holds the factory seal in place against the downward pressure exerted by the inner nozzle assembly.

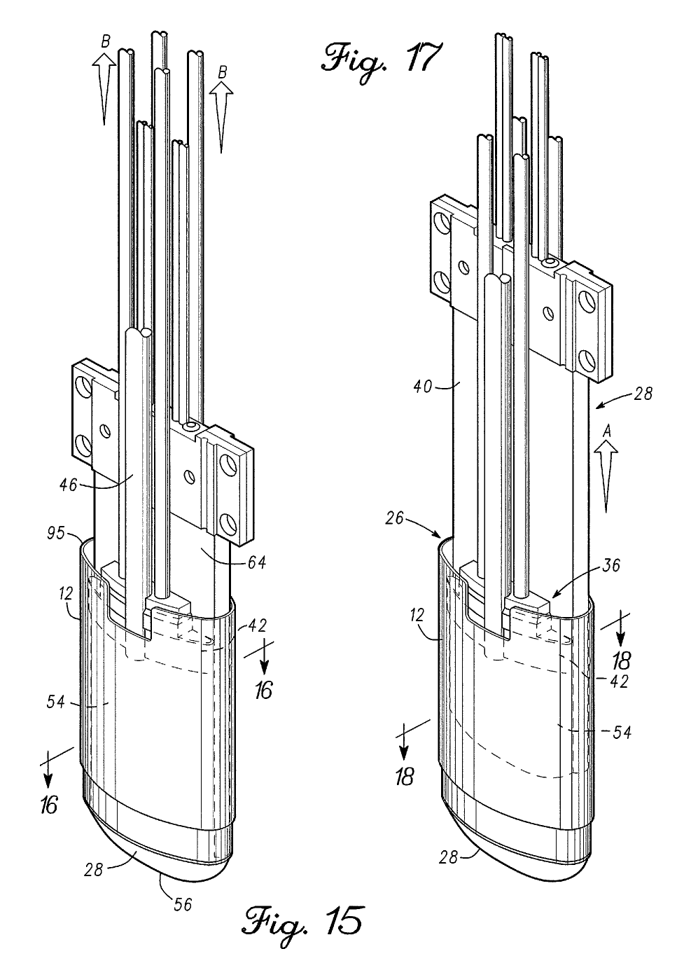

Next, as shown in FIGS. 15 and 16, the first portion, in liquid form (herein “the first fluid portion”), of the antiperspirant product is delivered to the container 12 to the open spaces on both sides of housing 54. Accordingly, to deliver the first fluid portion to the container in liquid form, the first fluid portion can be maintained at a temperature in the range of about 75 to about 80° C., for example, about 75° C. The first fluid portion 76 is delivered from delivery tubes 50 of outer nozzle assemblies 36 and 38, while the outer nozzle assemblies 36 and 38 are being simultaneously raised, as indicated by the arrows B. During delivery of the first fluid portion, the inner nozzle assembly 40 is maintained in its lowered position so that leading edge 56 provides a seal against the inner surface 74 of factory seal 28 to prevent first fluid portion 76 from flowing under the leading edge 56 and so that the outer surface 64 of the housing 54 provides a molding surface. Sealing is provided by the curved surface of the leading edge 56, which corresponds closely to the curvature of the surface 74 of the factory seal 28. Sealing can be enhanced by applying downward pressure to the inner nozzle assembly 40 during delivery of the first portion 16. The first fluid portion is molten, so that it is sufficiently fluid for delivery, but will solidify quickly as it cools. Because the outer surface 64 is chilled, the first fluid portion 76 will solidify relatively quickly.

The container 12, filled with the first fluid portion 76, is shown in FIG. 16. After the container 12 has been filled to a desired level, the first fluid portion 76 is allowed to solidify sufficiently so that a skin or thin solid layer will form to prevent the first fluid portion 76 from mixing with a second fluid portion. A skin thickness of from about 1 to about 2 millimeters (mm) is generally sufficient, typically requiring a dwell time of from about 1 to about 10 seconds, preferably from about 2 to about 6 seconds at about −10 to +20° C. The skin will form adjacent the surface 64 of the housing 54 due to the chilling of the surface 64. The dwell time will depend on the temperature to which the surface 64 of the housing 54 is cooled, and the temperature of the molten material when it is delivered. The resulting two regions 78 and 80 of the first fluid portion 76 (FIG. 12) will define the regions having the surfaces 20 of first portion 16 (FIG. 1).

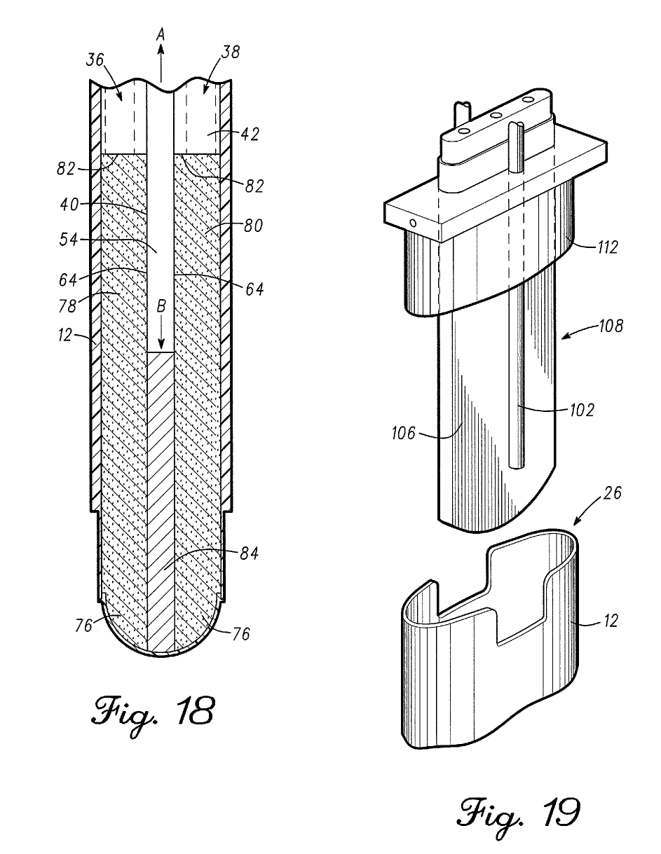

During this dwell time, and then during the subsequent filling step described below, the outer nozzle assemblies 36 and 38 are maintained in a position, shown in FIG. 18, at which a lower surface 81 of each scraper body 42 is in contact with the top surface of the regions 78 and 80 of the first fluid portion 76. In this position, the chilled scraper bodies serve several functions: (a) they aid in solidification of the top surface of the first fluid portion 76, (b) they hold the first fluid portion 76 in the container during the next step, described below, and (c) they scrape the outer surface 64 of housing 54 during the next step, which helps the solidified skin to release from surface 64. With respect to the first function, the chilling of the top surface of the first fluid portion 76 causes a skin to form at the top surface, which extends from the inner wall of the container and thus provides lateral support to the regions 78 and 80, preventing them from collapsing or leaning inward. If additional lateral support is desired, the upward movement of the outer nozzle assemblies during the filling process can be interrupted, e.g., for about one second, at an intermediate position within the container. The intermediate position may be about halfway up. This brief pause in the filling operation will allow a skin to form under the scraper bodies 42 at this point, providing additional lateral support to the regions 78 and 80.

Referring to FIGS. 17 and 18, when the first fluid portion 76 has sufficiently solidified (formed a skin), the inner nozzle assembly 40 is moved upwards (arrow A), out of the container. As the inner nozzle assembly 40 moves upward, a second fluid portion 84 is delivered to the space that becomes available between regions 78 and 80 of the first fluid portion 76 as the housing 54 is removed, as indicated by the arrow B in FIG. 18. The second fluid portion 84 is delivered through delivery channels 58 of the inner nozzle assembly 40. The second fluid portion will define the second portion 18 of the finished product (FIG. 1).

The retraction of the inner nozzle assembly 40 is coordinated with the delivery of the second fluid portion 84 so that the volume vacated by the nozzle is immediately filled with the liquid volume that is being delivered. This prevents damage to the weak skin that supports the regions 78 and 80 and prevents intermingling of the fluid portions. This coordination may be achieved, e.g., by electronically linking servo motors that control a delivery pump to a screw that retracts the nozzle assembly.

During delivery of second fluid portion 84, the chilled scraper bodies prevent the regions 78 and 80 from being lifted upwards by friction exerted by the outer surface 64 of housing 54, helping the skin to release from the surface 64, and scraping off any of the first fluid portion 76 that adheres to outer surface 64 during removal of housing 54. This leaves the outer surface 64 of housing 54 clean prior to filling of a new container.

The steps shown in FIGS. 17 and 18 complete the molding process and the manufacturing of the antiperspirant composition 25 illustrated in FIG. 3. Solidification of the first and second fluid portions is completed by cooling the product, for example, by passing the filled container through a forced air tunnel operating at between about 10 to about 25° C. The finished product (FIG. 3) is completed by sealing the open opposite end 26 with a package base (not shown) that includes advancement device 32.

Suitable materials for housing 54 include metals such as stainless steel, aluminum alloys, copper or beryllium. Coated metals can also be used, e.g., stainless steel coated with titanium nitride, chromium, or electroless nickel with a polytetrafluoroethylene (PTFE) infusion; aluminum coated with aluminum oxide hardcoat anodizing, hardcoat anodizing with a PTFE infusion, or electroless nickel with or without a PTFE infusion; or aluminum plated with nickel or chrome. The housing may be coated with a release coating such as liquid silicone to enhance release of the skin.

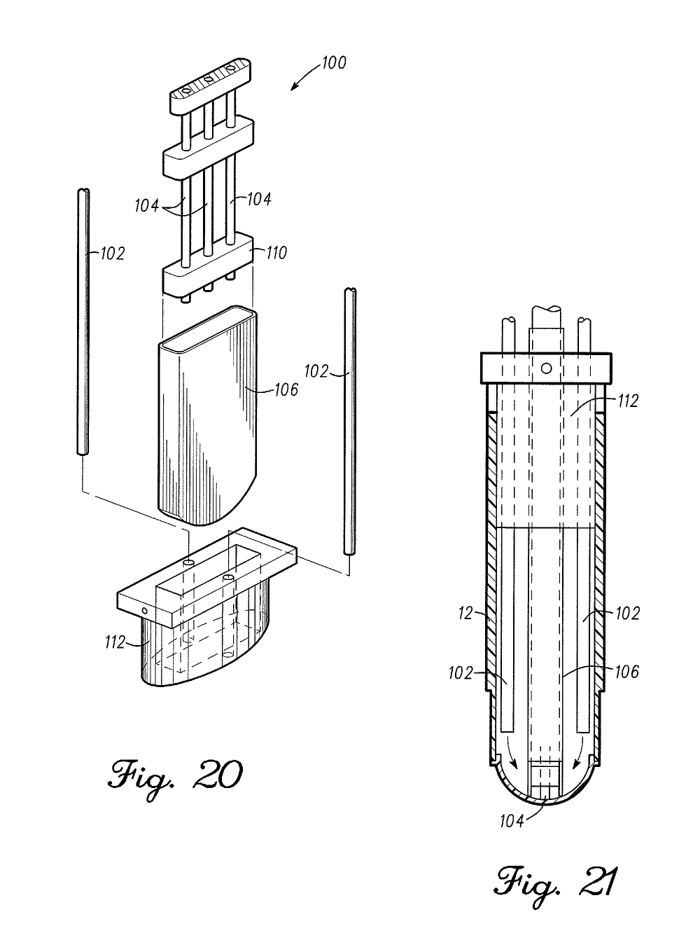

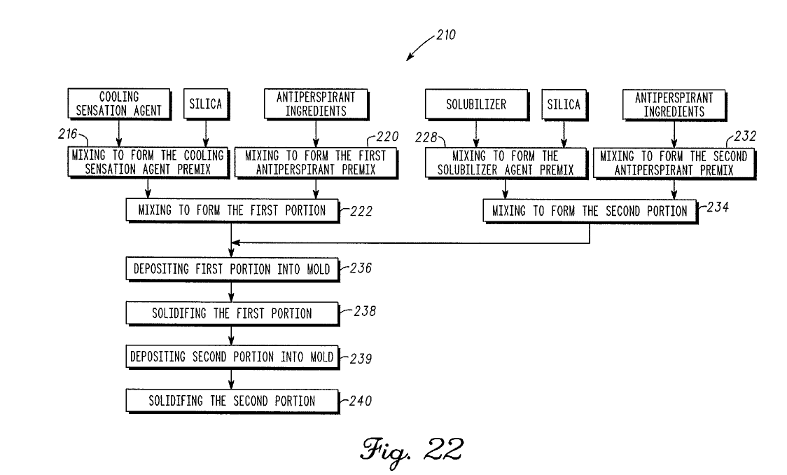

An alternative molding process can be performed using the filling assembly 108 shown in FIGS. 19 and 20. In this embodiment, the inner nozzle assembly 100 includes a plurality of delivery tubes 104 (FIG. 20) surrounded by a housing 106 that can be raised and lowered independently of the delivery tubes 104. The outer nozzle assemblies discussed above are replaced by the outer nozzles 102 and the scraper block 112, with the scraper block 112 performing the functions described above with reference to the scraper bodies 42. (If desired, the outer nozzle assemblies discussed above may be used in this embodiment.)

Because, in this embodiment, the housing 106 can be moved independently of the delivery tubes 104, the first and second fluid portions can be filled in any desired order, or simultaneously. If they are filled simultaneously, as shown in FIG. 21, the housing 106 would be left in place for a sufficient length of time to allow at least one of the compositions to form a skin to prevent intermingling of the two fluid portions. Thus, the housing 106 may be moved upward slowly, a few seconds behind the nozzles.

In this embodiment, it is generally useful that the inside surface of the housing 106 be scraped. To accomplish this, the member 110 (FIG. 20) is mounted on the delivery tubes 104, and shaped to closely fit the interior of the housing 106 so that, when the delivery tubes 104 are moved vertically relative to the housing 106, the member 110 will scrape the inner surface of the housing.

The following is an example of the first and second portions of the antiperspirant product in accordance with the present invention with each of the components set forth in weight percent. The example is provided for illustration purposes only and is not meant to limit the various embodiments of the antiperspirant product in any way. All materials are set forth in weight percent.

### Example 1

### First Portion

| Ingredient | Wt. % |
|---|---|
| Cyclopentasiloxane | 32.0 to 46.0 |
| Stearyl Alcohol | 15.0 to 21.0 |
| Aluminum Zironium | 18.0 to 24.0 |
| Pentachlorohydrex | |
| PPG-14 Butyl Ether | 9.0 to 15.0 |
| Hydrogenated Castor Oil | 2.0 to 5.0 |
| Myristal Myristate | 1.0 to 4.0 |
| Silica | 0.25 to 3.5 |
| Silica Dimethyl Silylate | 0.25 to 5.0 |
| Winsense Extra ® 500 | 0.10 to 3.0 |
| Water | 0.05 to 0.50 |
| Ethanol | 0.10 to 1.5 |
| Sipernat ® 22S | 0.50 to 3.0 |

### Second Portion

| Ingredient | Wt. % |
|---|---|
| Cyclopentasiloxane | 32.0 to 46.0 |
| Stearyl Alcohol | 15.0 to 21.0 |
| Aluminum Zironium | 18.0 to 24.0 |
| Pentachlorohydrex | |
| PPG-14 Butyl Ether | 9.0 to 15.0 |
| Hydrogenated Castor Oil | 2.0 to 5.0 |
| Myristal Myristate | 1.0 to 4.0 |
| Silica | 0.25 to 3.5 |
| Silica Dimethyl Silylate | 0.25 to 5.0 |
| Polyethylene Glycol | 0.5 to 5.0 |
| Polypropylene Glycol | 0.2 to 3.5 |
| Water | 0.2 to 4.0 |
| Sipernat ® 22S | 1.0 to 4.0 |
| Parfum | 0.1 to 6.0 |

### Example 2

### First Portion

| Ingredient | Wt. % |
|---|---|
| Cyclopentasiloxane | 32.0 to 46.0 |
| Stearyl Alcohol | 15.0 to 21.0 |
| Aluminum Zironium | 18.0 to 24.0 |
| Pentachlorohydrex | |
| PPG-14 Butyl Ether | 9.0 to 15.0 |
| Hydrogenated Castor Oil | 2.0 to 5.0 |
| Myristal Myristate | 1.0 to 4.0 |
| Silica | 0.25 to 3.5 |
| Silica Dimethyl Silylate | 0.25 to 5.0 |
| Menthol | 0.10 to 3.0 |
| Vanillyl Butyl Ether | 0.05 to 0.50 |
| Water | 0.05 to 0.50 |
| Ethanol | 0.10 to 1.5 |
| Sipernat ® 22S | 0.50 to 3.0 |

Accordingly, antiperspirant products that exhibit antiperspirant efficacy and that also provide a cooling sensation effect that is perceived by the user have been described. The various embodiments of the antiperspirant products comprise a first portion and a second portion. The first portion comprises a dispersed cooling sensation agent and the second portion comprises a solubilizer. By keeping the dispersed cooling sensation agent and the solubilizer separate in each of their corresponding portions, the cooling sensation effect is better preserved. The first portion, the second portion, or both portions of the antiperspirant product can provide antiperspirant efficacy. Upon application, the two portions combine to provide a user with an antiperspirant product that exhibits a cooling sensation effect throughout the day when it is needed most, e.g. when the user perspires.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A personal care product comprising:
a container; and
an antiperspirant product housed within the container, the antiperspirant product comprising:
a first portion comprising a dispersed cooling sensation agent; and
a second portion that is in non-mutual spatial relationship with the first portion and has a composition different from the first portion, the second portion comprising a solubilizer effective to solubilize the dispersed cooling sensation agent within body generated moisture and thereby impart to a user a cooling sensation effect.

2. The personal care product according to claim 1, wherein the dispersed cooling sensation agent comprises menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, or a mixture thereof.

3. The personal care product according to claim 1, wherein the first portion comprises the dispersed cooling sensation agent in an amount of from about 0.50 to about 3.0 wt. %.

4. The personal care product according to claim 1, wherein the solubilizer comprises polyglycol, polyethylene glycol, polypropylene glycol, or a mixture thereof.

5. The personal care product according to claim 1, wherein the second portion comprises the solubilizer in an amount of from about 0.7 to about 8.5 wt. %.

6. The personal care product according to claim 1, wherein the dispersed cooling sensation agent is adsorbed to surfaces of a first plurality of silica particles that are dispersed throughout the first portion.

7. The personal care product according to claim 6, wherein the first plurality of silica particles are porous hydrophilic silica particles, and the dispersed cooling sensation agent is one of adsorbed onto and absorbed into the porous hydrophilic silica particles.

8. The personal care product according to claim 6, wherein the first plurality of silica particles are effective to release the dispersed cooling sensation agent when contacted with the body generated moisture.

9. The personal care product according to claim 6, wherein the first portion comprises the first plurality of silica particles in an amount of from about 1.0 to about 4.0 wt. %.

10. The personal care product according to claim 1, wherein the solubilizer is adsorbed to surfaces of a second plurality of silica particles that are dispersed throughout the second portion.

11. The personal care product according to claim 10, wherein the second plurality of silica particles are porous hydrophilic particles, and the solubilizer is one of adsorbed onto and absorbed into the porous hydrophilic silica particles.

12. The personal care product according to claim 10, wherein the second plurality of silica particles are effective to release the solubilizer when contacted with the body generated moisture.

13. The personal care product according to claim 10, wherein the second portion comprises the second plurality of silica particles in an amount of from about 0.5 to about 3.0 wt. %.

14. The personal care product according to claim 1, wherein the first portion comprises a first region and a second region and the second portion is positioned between the first region and the second region.

15. An antiperspirant product comprising:
a first portion comprising a dispersed cooling sensation agent adsorbed on surfaces and absorbed into pores of a first plurality of silica particles that are dispersed throughout the first portion;
a second portion that is in non-mutual spatial relationship with the first portion and has a composition different from the first portion, the second portion comprising a solubilizer adsorbed on surfaces and absorbed into pores of a second plurality of silica particles that are dispersed throughout the second portion, the solubilizer effective to solubilize the dispersed cooling sensation agent within body generated moisture and thereby impart to a user a cooling sensation effect; and
wherein the first and second plurality of silica particles are effective to release the dispersed cooling sensation agent and the solubilizer when contacted with the body generated moisture.

16. The antiperspirant product according to claim 15, wherein the dispersed cooling sensation agent comprises menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, or a mixture thereof, and the solubilizer comprises polyglycol, polyethylene glycol, polypropylene glycol, or a mixture thereof.

17. The antiperspirant product according to claim 15, wherein the first portion comprises the dispersed cooling sensation agent in an amount of from about 0.50 to about 3.0 wt. %, and the second portion comprises the solubilizer in an amount of from about 0.7 to about 8.5 wt. %.

18. The antiperspirant product according to claim 15, wherein one of first and second portions contains an amount of unbound cooling sensation agent or unbound solubilizer that are not captured on either one of the first and second plurality of silica particles.

19. A method for manufacturing an antiperspirant product, the method comprising the steps of:
depositing a first portion of the antiperspirant product into a mold and allowing the first portion to at least partially solidify, the first portion comprising a dispersed cooling sensation agent; and
depositing a second portion of the antiperspirant product into the mold and allowing the second portion to at least partially solidify, the second portion having a composition different from the first portion and comprising a solubilizer effective to solubilize the dispersed cooling sensation agent within body generated moisture and thereby impart to a user a cooling sensation effect.

20. The method according to claim 19, further comprising forming the first portion including:
mixing a cooling sensation agent and a first plurality of silica particles together to form a cooling sensation agent premix;
mixing antiperspirant ingredients together to form an antiperspirant premix; and
mixing the cooling sensation agent premix and the antiperspirant premix together to form the first portion.

21. The method according to claim 20, wherein the step of mixing the cooling sensation agent premix and the antiperspirant premix together includes forming the first portion comprising the dispersed cooling sensation agent in an amount of from about 0.50 to about 3.0 wt. % and the first plurality of silica particles in an amount of from about 1.0 to about 4.0 wt. %.

22. The method according to claim 19, further comprising forming the second portion including:
mixing the solubilizer and a second plurality of silica particles together to form a solubilizer agent premix;
mixing antiperspirant ingredients together to form an antiperspirant premix; and
mixing the solubilizer agent premix and the antiperspirant premix together to form the second portion.

23. The method according to claim 22, wherein the step of mixing the solubilizer agent premix and the antiperspirant premix together includes forming the second portion comprising the solubilizer in an amount of from about 0.7 to about 8.5 wt. % and the second plurality of silica particles in an amount of from about 0.5 to about 3.0 wt. %.

24. The method according to claim 19, wherein the step of depositing a first portion comprises depositing a first portion comprising a dispersed cooling sensation agent selected from the group consisting of menthol, vanillyl butyl ether, peppermint oil, methane carboxamide ethyl pyridine, menthoxypropanediol, menthanediol, cyanomethylphenyl methane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, and mixtures thereof, and wherein the step of depositing a second portion comprises depositing a second portion comprising the solubilizer selected from the group consisting of polyglycol, polyethylene glycol, polypropylene glycol, and mixtures thereof.

25. The method according to claim 19, wherein the step of depositing the first portion of the antiperspirant product into the mold includes depositing the first portion into a container that will be used to store and dispense the antiperspirant product.
